(19) United States
(12) Plant Patent Application Publication
(10) Pub. No.: US 2003/0126661 P1
(43) Pub. Date: Jul. 3, 2003

(54) CHRYSANTHEMUM PLANT NAMED '92-333-2'

(76) Inventors: Neil Owen Anderson, St. Paul, MN (US); Peter David Ascher, St. Paul, MN (US)

Correspondence Address: WOOD,PHILLIPS,CATZ,CLARK & MORTIMER, CITICORP CENTER, 500 WEST MADISON STREET, SUITE 3800, CHICAGO, IL, 60661-2511, US

(21) Appl. No.: 09/503,271

(22) Filed: Feb. 14, 2000

(51) Int. Cl. A01H 5/00

(52) U.S. Cl. PLT/298

(57) ABSTRACT

A new and distinct Chrysanthemum plant named 92-333-2 is provided. This new cultivar was the result of a unique cross between Dendranthema weyrichii and Dendranthema grandiflora.

### BACKGROUND OF THE INVENTION

The present invention comprises a new and distinctive chrysanthemum plant, hereinafter referred to by the cultivar name 92-333-2. This new cultivar was the result of a cross in 1989 between Dendranthema weyrichii and Dendranthema grandiflora. More specifically, the breeding program which resulted in the production of the new cultivar was carried out at St. Paul, Minn. The female or seed parent of 92-333-2 was a Dendranthema weyrichii, commercially available from White Flower Farms, Connecticut having the following characteristics: (a) the plant habit is prostrate and the plant spreads via rhizomes to form a large mat after the first year; (b) the plant dimensions are that the plant has a diameter of about 1.5′ and is about 5-6″ tall; (c) the plant is hardy in zones 4-9 (Southeast)/Zone 10 (west); (d) the flower of the plant is a single daisy, having light lavender-colored ray florets and central disc florets with yellow pollen; (e) the plant has leaves that are dark green in color, with a very shiny leaf surface (glossy), and glabrous leaf margins that are deeply incised; and (d) the plant tends to rosette, needs cold treatment to flower consistently, flowering can be sporadic with gaps in the plant architecture and the plant is an obligate short-day plant. The male or pollen parent of 92-333-2 was a Dendranthema grandiflora which is commercially available from Yoder Brothers, Inc., Barberton, Ohio having the following characteristics: (a) the plant habit is cushion; (b) the plant dimensions are that the plant is similar to other cushion types commercially available from Yoder Brothers, Inc., such as, but not limited to the variety, ‘Raquel’; (d) the plant is hardy in zones 6-9 (Southeast)/Zone 10 (west); (d) the flower is a single or duplex daisy, possibly orange or bronze ray florets, central disc florets with yellow pollen; (e) the plant has leaves that are similar to other Yoder Brothers, Inc. cushion series chrysanthemums; and (d) the plant is a facultative short-day plant. The resulting seed, identified as 90-287-16 was collected. In 1991, a plant of 90-287-16 was crossed as the male parent with plants identified as 90-147-10, a University of Minnesota inbred parental selection, as the female parent and the resulting seeds, identified as cross number 92-333 were collected. In 1992, plants of 92-333 were germinated and flowering progeny evaluated. 92-333-2 was the second plant from the cross and was selected during the fall of 1992. The parentage of the new cultivar can be summarized as follows:

Dendranthema weyrichii×Dendranthema grandiflora

Asexual reproduction of the new cultivar by terminal or stem cuttings taken 1993 through 1999 at St. Paul, Minn., U.S.A. has demonstrated that the characteristics of the new cultivar as herein described are firmly fixed and are retained through successive generations of such asexual propagation.

### SUMMARY OF INVENTION

It was found that the cultivar of the present invention:

(a) exhibits extreme hybrid vigor,

(b) develops, in its second and subsequent years after planting, when grown in the fall under natural daylength and without the application of growth regulators, into a flowering herbaceous shrub having a plant height of from about 3.0 to about 3.75 feet and a spread from about 3.7 to about 6.0 feet,

(c) exhibits, in its second and subsequent years after planting and during the fall season (August-October), a massive floral display,

(d) displays flowers which are slightly toned with grey, giving the flower petals a slightly altered coloration,

(e) exhibits superior winter hardiness, including frost tolerance,

(f) exhibits self-pinching, and

(g) exhibits a less facultative, short-day response.

The 92-333-2 cultivar has not been observed under all possible environmental conditions to date. Accordingly, it is possible that the phenotype may vary somewhat with variations in the environment, such as temperature, light intensity, and day length.

When the new cultivar of the present invention is compared to ‘Empire Salsa’ (U.S. Plant Pat. No. 9,578, it is found to exhibit a more spreading and prolific habit accompanied with a massive floral display in its second and subsequent years after planting. Reference is made to attached Chart A which compares certain characteristics of 92-333-2 to ‘Empire Salsa’.

### BRIEF DESCRIPTION OF THE PHOTOGRAPHS

The accompanying photographs show as nearly true as it is reasonably possible to make the same in color illustrations of this type, typical flower and foliage characteristics of the new cultivar. The plants were grown in a greenhouse at St. Paul, Minn., USA.

FIG. 1 shows an adaxial and abaxial views of the leaf shape of chrysanthemum variety 92-333-2.

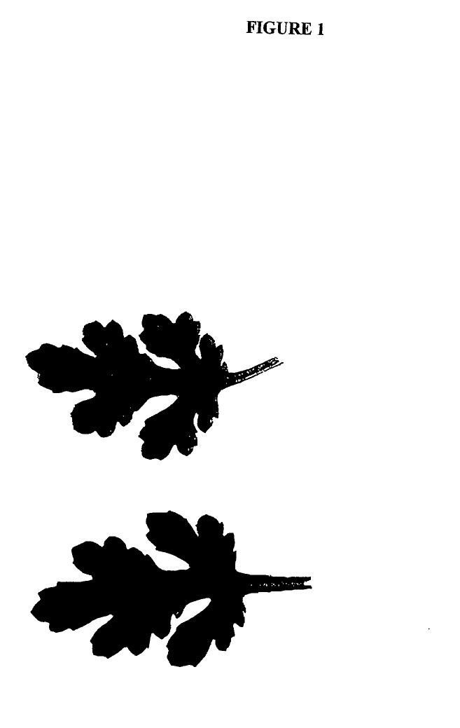

FIG. 2 shows the breeding history of chrysanthemum variety 92-333-2.

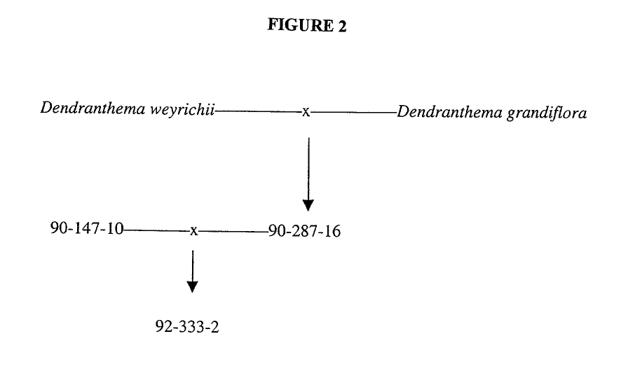

FIG. 3 is a color photograph of chrysanthemum variety 92-333-2 after one year of growth.

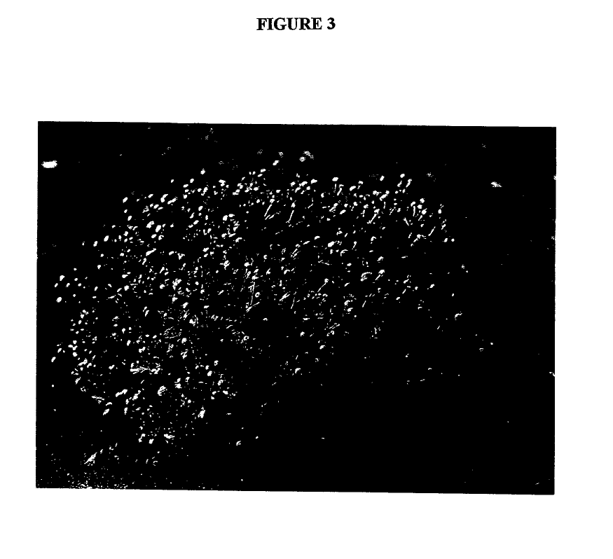

FIG. 4 is a color photograph of chrysanthemum variety 92-333-2 after two years of growth.

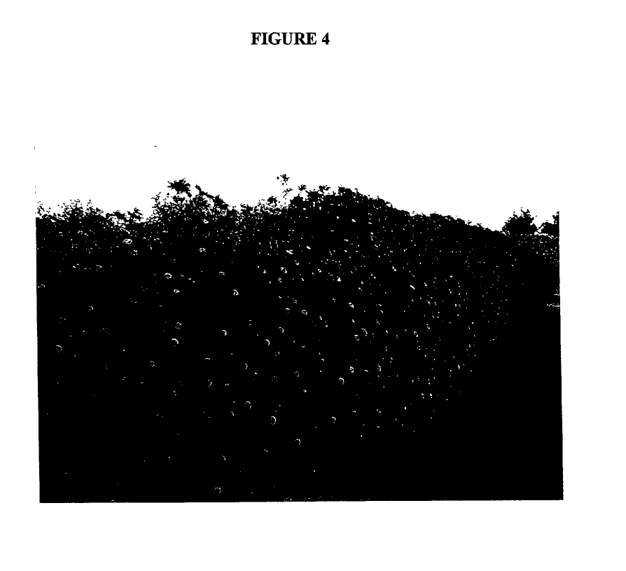

### DETAILED BOTANICAL DESCRIPTION

The chart used in the identification of colors described herein is the R.H.S. Colour Chart of The Royal Horticultural Society, London, England. The color values were determined on Oct. 15, 1999 in St. Paul, Minn. The readings were taken between 1:00 and 3:00 p.m. under approximately 2500 footcandles of light. The plants were produced from cuttings taken from stock plants and were grown under greenhouse conditions comparable to those used in commercial practice while utilizing a soilless growth medium and maintaining temperatures of approximately 72° F. during the day and approximately 65° F. during the night.

Propagation:

Type.—Herbaceous stem cutting.

Time to rooting.—About 1 week.

Rooting habit.—Vigorous.

Botanical Classification: Dendranthema×hybrida

Commercial Classification: Chrysanthemum hybrid

Plant Description:

Appearance, shape.—Mounded, spherical.

Appearance, growth habit.—Mound.

Appearance, growth rate/vigor.—Vigorous.

Plant height.—about 1.8 to about 2.0 feet (first year) about 3.0 to about 3.75 feet (second year).

Lateral branch length.—0.5 to 3 feet.

Quantity of lateral branches after removal of apical meristem.—One per node.

Stem color.—RHS Nickel Green 57/3.

Foliage Description:

Number of leaves per plant.—Greater than about 3,000.

Number of leaves per lateral branch.—3 to 25.

Leaf arrangement.—Alternate.

Leaf size, fully expanded, length.—5 cm.

Leaf size, fully expanded, width.—4 cm.

Leaf apex.—Acute.

Leaf base.—Truncate to auriculate.

Leaf margin.—Incised.

Leaf texture.—Glaucous/hirsute.

Petiole length.—3 to 5 cm.

Color, young foliage adaxial surface.—RHS Nickel Green 57/1.

Color, young foliage abaxial surface.—RHS Nickel Green 57/3.

Color, fully expanded foliage adaxial surface.—RHS Paris Green 58/1.

Color, fully expanded foliage abaxial surface.—RHS Paris Green 58/2.

Color, venation adaxial surface.—RHS Nickel Green 57/3.

Color, venation abaxial surface.—RHS Nickel Green 57/3.

Color, petiole.—RHS Nickel Green 57/2.

lnflorescence Description:

Appearance.—Head (composite); duplex/triplex daisy.

Flowering.—response 6 weeks (SD).

Quantity of inflorescences.—About 5,000 (second year).

Inflorescence size, diameter.—6 to 9 cm.

Inflorescence size, depth (height).—2 to 3 cm.

Inflorescence size, diameter of disc.—1.2 to 3 cm.

Opening inflorescences, bud shape.—Upright, tubular.

Opening inflorescences, bud size, length.—1.75 to 2 cm.

Opening inflorescences, bud size, width.—1.25 to 1.8 cm.

Opening inflorescences, bud color.—RHS Turkey Red 721/3.

Ray florets, shape.—Daisy.

Ray florets, size, length.—2.1 to 3 cm.

Ray florets, size, width.—0.75 to 0.9 cm.

Ray florets, apex.—Obtuse.

Ray florets, base.—Cuneate.

Ray florets, margin.—Entire.

Ray florets, texture.—Glabrous.

Ray florets, aspect.—Horizontal to slightly pendant.

Number of ray florets per inflorescence.—About 68 to about 72.

Ray florets, color, when opening, adaxial surface.—RHS Erythrite Red oo27.

Ray florets, color, when opening, abaxial surface.—RHS Erythrite Red oo27/1.

Ray florets, color, mature, adaxial surface.—RHS Erythrite Red oo27.

Ray florets, color, mature, abaxial surface.—RHS Peony Purple 729/3.

Ray florets, color, fading to.—RHS Turkey Red 721/3.

Disc florets, shape.—Tubular, rounded at tip.

Disc florets, size, length.—0.4 cm (unopened) 0.4 cm (opened).

Disc florets, size, width.—0.1 cm (unopened) 0.2 cm (opened).

Number of disc florets per inflorescence.—About 120.

Disc florets, color, immature.—RHS Chrome Yellow 605/2.

Disc florets, color, mature.—RHS Chrome Yellow 605.

Peduncle, aspect, strength.—Stiff.

Peduncle, aspect, angle to stem.—45°.

Peduncle, length, first peduncle.—3.2 cm.

Peduncle, length, fourth peduncle.—8.4 cm.

Peduncle, texture.—Ribbed, mildly hirsute.

Peduncle, color.—RHS Nickel Green 57/2.

Reproductive organs, androecium, floret location.—Disc florets.

Anther color.—RHS Chrome Yellow 605.

Pollen, abundance.—Abundant.

Pollen, color.—RHS Chrome Yellow 605.

Reproductive organs, gynoecium, floret location.—Disc/ray florets.

Style color.—RHS Chrome Yellow 605/3.

Disease Resistance: None Known

Seed Production: About 192 ovules/flower

Winter Hardiness: Hardy in zones 3-10 in uncovered field conditions without the need for added protection such as snow fences, mulch, etc.

Frost Tolerance: Yes, extends blooming season to the first freeze in the north (In zones 3-4 the first frost usually takes place between September 1-15. In zones 3-4, the first freeze usually takes place between October 1-20).

What is claimed is:

1. A new and distinct cultivar of Chrysanthemum named 92-333-2 substantially as herein shown and described, which:
(a) exhibits extreme hybrid vigor,
(b) develops, in its second and subsequent years after planting, when grown in the fall under natural daylength without the application of growth regulators, into a flowering herbaceous shrub having and without the application of growth regulators, into a flowering herbaceous shrub having a plant height of from about 3.0 to about 3.75 feet and a spread from about 3.7 to about 6.0 feet,
(c) exhibits, in its second and subsequent years after planting and during the fall season (August-October), a massive floral display,
(d) displays flowers which are slightly toned with grey, giving the flower petals a slightly altered coloration,
(e) exhibits superior winter hardiness, including frost tolerance,
(f) exhibits self-pinching, and
(g) exhibits a less facultative, short-day response.
